(19) United States
(12) Patent Application Publication
(10) Pub. No.: US 2006/0269612 A1
(43) Pub. Date: Nov. 30, 2006

(54) Intracellular thermal ablation using nano-particle electron spin resonance heating

(75) Inventors: Xiao Dong Xiang, Danville, CA (US); Haitao Yang, Albany, CA (US)

Correspondence Address: QUINE INTELLECTUAL PROPERTY LAW GROUP, P.C., P O BOX 458, ALAMEDA, CA, 94501, US

(73) Assignee: Intematix Corporation

(21) Appl. No.: 11/410,512

(22) Filed: Apr. 24, 2006

Related U.S. Application Data
(60) Provisional application No. 60/673,945, filed on Apr. 22, 2005.
(60) Provisional application No. 60/673,944, filed on Apr. 22, 2005.

(51) Int. Cl. A61K 33/26; A61K 9/14

(52) U.S. Cl. 424/489; 424/646; 977/906

(57) ABSTRACT

This invention pertains to the use of spin resonance absorption heating as a therapeutic treatment method wherein electron spin resonance absorption of superparamagnetic (SPM) nanoparticles can be used as an intracellular heating method, more preferably as an in vivo heating method that can be utilized in a variety of therapeutic contexts and can further allow for resonance imaging and internal thermometry.

### CROSS-REFERENCE TO RELATED APPLICATIONS

This application claims benefit of and priority to U.S. Ser. No. 60/673,945, filed on Apr. 22, 2005, which is incorporated herein by reference in its entirety for all purposes.

This application claims benefit of priority from provisional application 60/673,944 filed 22 Apr. 2005 and from U.S. patent application Ser. No. ______ filed 21 Apr. 2006 and titled MRI TECHNIQUE BASED ON ELECTRON SPIN RESONANCE AND ENDOHEDRAL CONTRAST AGENT, Atty. Docket No. 318-003010US.

This application incorporates by reference U.S. patent application U.S. Ser. No. 10/835,247 titled SPIN RESONANCE HEATING AND/OR IMAGING IN MEDICAL APPLICATIONS, filed on Apr. 28, 2003, Atty. Docket No. 318-001110U.S. and which claims benefit of US Ser. No. 60/466,099, filed on Apr. 28, 2003 both incorporated herein by reference in their entirety for all purposes.

This application incorporates by reference U.S. patent application U.S. Ser. No. 11/351,312 titled ENDOHEDRAL FULLERENES AS SPIN LABELS AND MRI CONTRAST AGENTS, Atty. Docket No. 318-002110US filed Feb. 8, 2006 and provisional application U.S. Ser. No. 60/652,288, Atty. Docket No. 318-002100US filed Feb. 10, 2005, both incorporated herein by reference in their entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention is related to a method of imaging and/or selective heating therapy using intracellular nano-sized superparamagnetic (SPM) particles. Upon application of an RF field in magnetic field, the particle can absorb the RF power by magnetic resonance and the energy is released as heat, which can selectively destroy the cells or tissues with the particles placed intracellularly. A magnetic field gradient can also be used to localize the heating region, to a smaller region than the region than particles are distributed. In specific embodiments internal thermometry and Imaging are also provided.

### BACKGROUND OF THE INVENTION

Electromagnetic radiation (e.g. X-ray and γ-ray from radioactive elements) with very high-energy photon particles has been traditionally used for the therapeutic treatment of certain diseases such as cancer. The high-energy radiation beam can be focused to a specific location, even deep within the body, to destroy the targeted cells. However, the normal cells at the same location will also simultaneously be killed. Consequently, there is always a conflict between the dosages that will effectively kill the disease cells and keep enough normal cells for recovery.

It is highly desirable if the radiation can be specifically targeted only to diseased cells at specific locations. It is also desirable that the radiation energy and dosage can be dramatically lowered for safety reasons. Consequently hyperthermia has been explored as a treatment tool for cancers, for other pathologies treated by inhibiting cell growth or proliferation, and for the cosmetic ablation of tissues.

It is known that elevating the temperature of tumors is helpful in the treatment and management of cancerous tissues. The mechanisms of selective cancer cell eradication by hyperthermia are not completely understood. Four cellular effects of hyperthermia on cancerous tissue have been proposed, (i) changes in cell or nuclear membrane permeability or fluidity, (ii) cytoplasmic lysomal disintegration, causing release of digestive enzymes, (iii) protein thermal damage affecting cell respiration and the synthesis of DNA or RNA and (iv) potential excitation of immunologic systems.

To reduce side effects and improve the effectiveness of chemotherapy and radiotherapy commonly used for cancer treatment, less invasive therapies including thermal ablation and hyperthermia have emerged as safer and more effective technologies. Hyperthermia is heating organs and tissues to temperatures between 41° C. and 46° C., which reduces the viability of cancer cells and increases their sensitivity to chemotherapy and radiotherapy [1-4]. Thermal ablation is heating tumors to higher temperatures, up to 56° C., causing necrosis, coagulation, or carbonization of the tumor cells [4].

Conventional hyperthermia techniques involve heating cancer cells from outside of the cells. Various methods ranging from hot baths, wax encasement, induced fevers, local perfusion of extremities with heated chemotherapeutic agents, diathermy, radio-frequency, microwave heating and ultra-sound heating have been used in the past [6, 7].

Clinical hyperthermia trials have generally focused on this so called “extracellular” approach and consist of three different domains: whole body hyperthermia, regional hyperthermia (RHT), and local hyperthermia (LHT). Successful LHT and RHT rely on targeting and directing the heat toward cancer cells with an accurate control of temperature distribution. LHT and RHT are commonly performed using radio-frequency, microwave, or ultrasound applicators. The state-of-the-art hyperthermia system is the annular phased array system (APAS), in which microwave antennae are arranged cylindrically around the axis of the body to focus the electromagnetic field on a region with a typical diameter of ˜10 cm (depending on the microwave frequency), an undesirably large area [11]. In addition, APAS for RHT of deeply seated tumors is limited by the known heterogeneity of tissue electrical conductivities or highly perfused tissues, which makes selective heating of those regions difficult [81.

### SUMMARY OF THE INVENTION

Hyperthermia (raising the temperature of a tumor to a range between 41° C. to 46° C.) is one of several methods to destroy cancer cells since malignant cells are found to be more sensitive to heat than normal cells [1-4]. Hyperthermia can be used either together with radiation therapy and chemotherapy to achieve therapeutic effects or by itself to shrink and even completely eradicate tumors [5]. Conventional hyperthermia techniques involve heating cancer cell from outside of the cell [6, 7].

Gordon et al. in 1979 suggested an “intracellular” approach using magnetic nano-particles (2-6 nm) as heating media [8]. It was found that nano-particles were predominately (specifically) ingested by the cancer cells rather than by normal cells. This observation was further confirmed in later studies by Jordon et al. [9]. The specific uptake of magnetic nano-particles by cancer cells raised the potential for specific killing of cancer cells. In fact, in Gordon et al.'s publication [8], evidence was found that cancer cells inside tumor have been killed while normal tissues around them remain alive and healthy, however there has been some debate around validity of “intracellular” heating effects.

The present invention involves techniques to achieve the long sought goal of “intracellular cancer thermal-therapy” using nano-particle ferromagnetic resonance for intracellular cancer thermal ablation therapy and also for internal thermometry. Some advantages of the techniques of the present invention include:

1) Heat absorption of magnetic nano-particles at ferromagnetic resonance biased by DC magnetic field is 10⁴-10⁶ times more efficient than that of previously adopted Néel heating in nano-particle hyperthermia techniques with AC magnetic field only.

2) Since ferromagnetic resonance occurs only when the applied local magnetic field and the electromagnetic radiation frequency satisfy the resonance conditions (similar to MRI or eMRI imaging), heating can be directed at and limited to a specific volume at specific location. As a result, only cells ingested with nano-particles at a specific location will be heated and killed.

3) The temperature dependence of ferromagnetic electron resonance frequencies of the nano-particles can be used as internal thermometry to monitor the temperature of the particles and cells to greatly increase the safety and reliability of this therapeutic technique.

4) Since the nano-particles can also serve as MRI imaging and/or eMRI contrast agents, MRI image guided surgical heating therapy can be realized.

In specific embodiments, the invention involves densely packed nano-particles (and optionally proteins) within cancer cells to heat those cells to a much higher temperature than the average temperature of the region, especially the temperature of normal cells with no nano-particle filling.

Similar to previous SPM Néel heating technique, surface charge of SPM particles can be optimized to further enhance the differential uptake ratio between cancer and normal cells [9]. In order to control ferromagnetic resonance frequency under 0.5-1 GHz, so that RF can penetrate and is safe to human body, selected compositions that give rise to lower saturation magnetization (and consequently lower ferromagnetic resonance frequency) will be used for nano-particle fabrication.

In further embodiments, the invention is involved with:
1) Synthesizing a series of Ga-YIG SPM nanoparticles with controlled size of about 10 nm using Internatix's proprietary combinatorial laser pyrolysis nano-particle synthesis technique;
2) Modifying the surface charge conditions of the nano-particles to suspend them in bio-compatible solution;
3) Use the ferromagnetic resonance properties of these nano-particles to evaluate their heating efficiency and compare it to theoretical calculation;
4) Use the temperature dependence of electron spin resonance frequency of candidate materials for internal thermometry applications.

In further embodiments, the invention is involved with methods that:
1) Synthesize Ga doped YIG SPM nano-particles suspended in bio-compatible solution with ferromagnetic resonance below 1 GHz and heat absorption efficiency at least one order of magnitude higher than Néel heating media.
2) Perform a non-invasive internal thermometry technique with temperature sensitivity of better than 1° C.
3) Use the intracellular heating techniques described herein to selectively destroy one or more cells for treatment or one or more diseases or conditions.
   1. Spin Resonance and Heating

Thus, in order to overcome the shortcomings of previously described techniques, this invention provides electron spin resonance heating methods for biomedical applications using intracellular nano-particles. Magnetic resonance (e.g., MRI) methods and nuclear spin resonance (e.g. NMR) methods have been proposed for hyperthermic treatment modalities. Spin resonance heating occurs when applied radiation field (microwave or RF) frequency, magnetic field and material's gyromagnetic ratio satisfy the following equation (Poole (1983) Electron Spin Resonance (2nd Edition), A Wiley-Interscience Pub.):
hv=gμ_{B}B 1
where h is Plank's constant, v the magnetic spin resonant frequency, B the external magnetic field, g the gyromagnetic ratio, and μ_{B} is Bohr magneton for electron spin resonance (ESR); for nuclear magnetic resonance (NMR), μ_{B} should be replaced by nuclear magneton μ_{N}. Nuclear spins or electron spins absorb photon energy at the spin resonance and jump to higher energy level precessing coherently. As the spin precessing relaxes through spin-lattice interaction, the absorbed electromagnetic energy turns into heat. The heat generation is proportional to the density of un-paired spin and spin population difference. The spin population difference in the two adjacent Zeeman levels is governed by Boltzmann statistics:

$$Δ n = 1 - exp ( - \frac{hv}{kT} ) \qquad 2$$

Since the energy difference between two Zeeman levels is small, at elevated temperatures, thermal excitation makes the spins almost equally occupy both energy levels leaving a very small fraction of spin to contribute to the spin resonance. At room temperature and in a 5T magnetic field; this corresponds to a factor of 10⁻⁵ reduction in resonance absorption for a typical NMR (or MRI). Therefore, nuclear spin resonance absorption is generally not effective in generating heat. In addition, nuclear spin resonance absorption heats up all protons, which may not be suitable for targeted therapeutic treatment.

Since the mass of an electron is about 1863 times smaller than a proton for electron spin resonance (ESR), the spin population difference for ESR is approximately 10⁻² at room temperature and a 5T magnetic field. The required frequency for electromagnetic radiation to excite the spin resonance using traditional approaches is much higher than that of NMR, typically 9.8 GHz. The radiation at this frequency cannot penetrate deeply and suffers a large dielectric loss in biological tissues that renders the technique useless in most cases. Recently, there has been much effort put forth to use lower frequency ESR technique (200 MHz to 3 GHz) for MRI. However, if we lower the frequency of ESR to the same frequency of NMR, it will have the same reduction of 10⁻⁵ in spin population difference, and therefore, resonance absorption the same as NMR.

This invention pertains to the use of spin resonance absorption heating as a therapeutic treatment method based on the discovery that electron spin resonance absorption of superparamagnetic (SPM) nanoparticles can be used as an effective heating method, more preferably as an in vivo heating method that can be utilized in a variety of therapeutic contexts including as an intracellular heating method.

The superparamagnetic nanoparticles according the present invention are introduced intracellularly to a desired target cell, tissue, organ, etc. thereby allowing selective heating of the target. Spatially resolved (localized) heating can also be provided by tailoring the magnetic field gradient during electron spin resonance (ESR) as described herein. Since spin resonance occurs only when the applied magnetic field and electromagnetic radiation energy satisfy certain resonance conditions, heating can be directed and limited only to the SPM particles at a specific location. As a result, only cells, and/or tissues, and/or organs, etc., that contain or are adjacent to the spatially selected particles will be heated and, if desired, damaged. Most of the normal cells will not be affected during the treatment.

In certain embodiments, mechanism, superparamagnetic particles are treated chemically to allow them to be selectively taken up by cells or tissues of interest. Because of the long-range spin-spin correlation in superparamagnetic materials, the spin population difference is nearly one in contrast to that in nuclear or electron paramagnetic spin resonance where the spin population difference is only 10⁻⁵. This makes resonance absorption at least 5 orders of magnitude higher than conventional NMR or ESR. As a consequence, spin resonance heating will be 5 orders of magnitude more effective and viable to realistic therapeutic applications. Since the superparamagnetic spin resonance is far away from the spin resonance of any cells in biological specimen under the same magnetic field, the absorption and conversion of electromagnetic energy to heat is highly selective only to the resonating SPM particles and the immediate vicinity. The other regions of the subject (e.g., a human body) can be spared of any harmful side effects.

Thus, in one embodiment, this invention provides composition for selectively heating (via electron spin resonance (ESR)) and/or imaging a cell, tissue, or organism. The composition comprises a superparamagnetic nanoparticle that optionally is chemically or electrically treated to be taken up by target cells. The superparamagnetic nanoparticle comprises a material that typically has an electron spin resonance (ESR) Q greater than 10, more preferably greater than 50 or 100, and most preferably greater than about 500. In certain embodiments, Q ranges from about 10 to 3000, more preferably from about 100 to about 1000. In certain embodiments, the superparamagnetic nanoparticle comprises a garnet or a spinel (e.g., a garnet or a spinel selected from Table 2. In certain embodiments, the superparamagnetic nanoparticle comprises yttrium ion garnet (YIG), more preferably substituted YIG (e.g. as shown in Table 2, or with aluminum, gallium, indium, ferrite, etc.). In certain embodiments, the superparamagnetic nanoparticle comprises gamma-Fe2O3. In certain embodiments, the SPN has at least one dimension less than about 500 nm, in certain embodiments, the SPN has no dimension greater than about 500 nm, and in certain embodiments, SPN has at least one dimension less than about 100 nm.

In another embodiment, this invention provides a composition for selectively heating or imaging a cell, tissue, or organ. The composition typically comprises superparamagnetic nanoparticles (e.g., any of the SPNs as described above) in a pharmacologically acceptable excipient.

In another embodiment, this invention provides a mixture of compositions each selected for one or more of (1) selectively heating a cell; (2) imaging a cell, tissue, or organ, or (3) providing internal thermometry in a cell, tissue or organ. The compositions typically comprise various superparamagnetic nanoparticles (e.g., any of the SPNs as described above) treated appropriately to have the desired physiological properties in a body.

Also provided is a method of selectively heating an organ, a cell, a tissue, a molecule, etc. The method typically involves introducing intracellularly into the cell, tissue, or molecule with a composition comprising a superparamagnetic nanoparticle (SPN) and heating the superparamagnetic nanoparticle using electron spin resonance. In certain embodiments, the electron spin resonance is at an RF ranging from about RF frequency ranging from about 200 to about 2,000 MHz MHz. In certain embodiments, the electron spin resonance is at an RF ranging from about 500 to about 1,000 MHz. In certain embodiments, the electron spin resonance is spatially localized by a magnetic field gradient over a region smaller than the region over which the superparamagnetic nanoparticles are distributed. The SPN includes, but is not limited to any of the SPNs described above.

In still another embodiment, this invention provides a selectively heating a cell, tissue, or organ. The method typically involves delivering a plurality of superparamagnetic nanoparticles intracellularly and heating the superparamagnetic nanoparticles using electron spin resonance. The method can be performed ex vivo, in vivo, and in situ. In certain embodiments, the superparamagnetic nanoparticles are delivered directly into the cell, tissue, or organ (e.g., by injection, via a catheter, during a surgical procedure, etc.). In certain embodiments, the superparamagnetic nanoparticles are delivered systemically administered to an organism. The SPNs include, but are not limited to any of the SPNs described above. In certain embodiments, the electron spin resonance is at an RF ranging from about 200 to about 2,000 MHz. In certain embodiments, the electron spin resonance is at an RF ranging from about 500 to about 1,000 MHz. The electron spin resonance can be spatially localized by a magnetic field gradient over a region smaller than the region over which the superparamagnetic nanoparticles are distributed. The method can, optionally, further involve imaging the cell, tissue, organ, or molecule (e.g., via thermography, MRI, ESR, x-ray, etc.). In various embodiments, the cell or tissue is a cancer cell.

In certain embodiments, this invention provides methods of selectively heating a cancer cell. The methods typically involve contacting a cancer cell with a superparamagnetic nanoparticle that is introduced or has been modified to be selectively taken up by the cell and performing electron spin resonance to heat the superparamagnetic nanoparticle. Suitable superparamagnetic nanoparticles (SPNs) are SPNs for electron spin resonance and include, but are not limited to any of the SPNs described herein (e.g., SPNs with a Q greater than 10, SPNs comprising a material in Table 2, etc.). The method can, optionally, further comprise imaging the cell, tissue or molecule preferably by detecting the SPN, e.g., via thermography, MRI, ESR, x-ray, etc. In certain embodiments, the chelate comprises DOTA.

In the thermal heating cancer therapy application, it's very important to have the capability to monitor the internal temperature change of the treated region non-invasively in the real time. Thus, the present invention according to specific embodiments employs temperature monitoring and imaging by detecting the temperature dependence of electron spin resonance properties, such as resonance frequency or relaxation time T₁/T₂.

In specific embodiments, the heating technique utilizes the electron spin resonance system, the same setup can be used to do the temperature monitoring and imaging without adding much extra efforts. The paramagnetic or ferromagnetic nano-particles with temperature dependence (frequency or relaxation time) will be mixed together with the heating particles as the temperature agents and taken by cancer cells. Thus, the spin resonance properties change of the temperature agents will reveal the cancer cell temperature change. The 3D imaging technique will be the same as conventional MRI technique, however, with much lower magnetic field (lower cost). The invention in specific embodiments can evaluate different temperature agent materials and different detection methods (frequency or relaxation time detection) to get the most reliable and sensitive results for particular applications.

This invention also provides kits for selectively heating (e.g., via ESR) or imaging a cell, tissue, organ, etc. The kit typically includes a container containing superparamagnetic nanoparticles (SPN) or a mixture thereof treated to be taken up by a biological target comprising the cell or tissue. The SPN includes, but is not limited to any of the SPNs described above. The SPN can be provided dried or suspended in a solution (e.g., a pharmacologically acceptable excipient).

In another embodiment, a kit is provided for selectively heating or imaging a cell or tissue. The kit typically includes a container containing a superparamagnetic nanoparticle where the nanoparticle is prepared for intracellular uptake. The kit can, optionally, further comprising instructional materials teaching the use of the superparamagnetic nanoparticles to selectively heat or image a cell or tissue. In certain embodiments, the superparamagnetic nanoparticle is specifically taken up by a cancer cell.

### Definitions

The term “nanoparticle”, as used herein refers to a particle having at least one dimension equal to or smaller than about 500 nm, preferably equal to or smaller than about 100 nm, more preferably equal to or smaller than about 50 or 20 nm, or having a crystallite size of about 10 nm or less, as measured from electron microscope images and/or diffraction peak half widths of standard 2-theta x-ray diffraction scans.

### BRIEF DESCRIPTION OF THE DRAWINGS

FIG. 1 is a diagram illustrating room temperature saturation magnetization of Ga-YIG as a function of Ga concentration according to specific embodiments of the invention.

FIG. 2 is a diagram illustrating temperature dependence of the longitudinal relaxation rates of N@C₆₀ according to specific embodiments of the invention.

FIG. 3 illustrates an example of an instrument set-up that can be used for characterization of particle spin resonance detection and heating and human body therapeutics according to specific embodiments of the invention.

FIG. 4 illustrates measured ferromagnetic resonances of three Gd-YIG spheres with different saturation magnetization according to specific embodiments of the invention.

FIG. 5 is a diagram of an example instrument setup for spin resonance detection according to specific embodiments of the invention. A and B illustrate RF coil and protection circuit design, where A shows surface R.F. coil, which is tuned to resonance with the tuning capacitor CT and matched to 50 ohms with a matching capacitor CM and B shows a circuit diagram for receiver isolation using a quarter wavelength cable and protection Zener diode.

FIG. 6 illustrates ferromagnetic resonance of YIG sphere measured by EMP according to specific embodiments of the invention.

FIG. 7A-B illustrate typical gradient coils used to generate field gradient along x, y, z directions according to specific embodiments of the invention. A and B illustrate generation of the magnetic field gradient. A:The x gradient is formed by a current that runs on a cylinder such that the two arcs above are both bringing current around the cylinder in a clockwise direction. The arcs shown below will bring current around the cylinder in a counter-clockwise direction. This creates a magnetic field pointing in the z direction that varies in strength along the x direction. For a y gradient, this configuration need only be rotated by 90°. B: A magnetic field gradient in the z direction is made by two circular coils whose currents run in opposite directions. This makes a magnetic field that points in the z direction and varies in strength along z.

FIG. 8 illustrates steps for magnetic nano-particles surface modification according to specific embodiments of the invention.

FIG. 9A-C illustrate (a) Nano-particle synthesizing system; (b) TEM image of the TiO2 Nano-particles prepared by CLP, inset is the HR image of the crystal structure; (c) TEM image of YIG nano-particles prepared by CLP, left image shows the crystal structure.

FIG. 10 illustrates schematics of nano-particle collector of combinatorial laser pyrolysis according to specific embodiments of the invention.

### DETAILED DESCRIPTION

2. Cellular Uptake and Intracellular Thermal Heating

Gordon et al in 1979 proposed an “intracellular” approach for thermal heating using magnetic nano-particles (2-6 nm) as heating media [8]. It was found that nano-particles were predominately (specifically) ingested by the cancer cells rather than by normal cells. This observation was further confirmed in later studies by Jordon et al. [9]. The specific uptake of magnetic nano-particles by cancer cells raised the potential for specific killing of cancer cells. It was further suggested by Gordon et al. (with a certain degree of evidence), that observed effect was due to that cell membranes shield the heat from conducting to the normal cells during electromagnetic field heating (later attributed to “Néel Heating” mechanism).

Over the past two decades, hyperthermia techniques based on this kind of super-paramagnetic (SPM) nanoparticles (nano-size single domain magnetic particles) have been extensively studied 19, 12-171. The fundamental heating mechanism of all these techniques is identified as Néel heating [18]. It is a more efficient heating mechanism than hysteresis heating in large magnetic particles. The mechanism behind Néel heating is that a small single domain magnetic particle can relax (re-orient) its magnetization direction polarized by an external magnetic field through a thermal process, i.e., the thermal energy is enough to re-orient the magnetization of a small magnetic domain.

Gordon et al's proposal of intracellular hyperthermia was recently “discredited” by Rabin through a mainly theoretical argument [19]. In Rabin's theoretical calculation, no thermal barrier was built in anywhere. If we assume there is no thermal barrier of the cell membrane as Rabin suggested, Rabin's calculation indicates that a single nano-particle or a single cell containing nano-particles cannot be heated to the temperature causing cell damage, while a larger volume of cells with a uniform distribution of nano-particles can be heated to high enough temperature to cause cell damage. However, the realistic tumor region (>1 mm³) contains cancer cells (˜5 μm) filled with a high concentration of nano-particles and normal cells (presumably the same size) with no filling.

The present invention according to specific embodiments extends on earlier work to use densely packed nano-particles and optionally proteins within cancer cells to heat those cells to a much higher temperature than the average temperature of the region, especially the temperature of normal cells with no nano-particle packing, in the collective heating process by many nano-particles with very non-uniform distribution. Even without the thermal barriers effect of membrane, “Intracellular” (though this does not mean single cellular) heating effect is used therapeutically as discussed herein.

That individual cells can maintain higher temperatures than there surroundings is supported by evidence found in [20]). Thermal Imaging of Receptor-Activated Heat Production in Single Cells Ofer Zohar,* Masayaki Ikeda,^{#} Hiroyuki Shinagawa,^{#} Hiroko Inoue,^{#} Hiroshi Nakamura,^{#} Danek Elbaum,^{§} Daniel L. Alkon,* and Tohru Yoshioka^{#}. This reference shows experimental observation of intracellular heat production in single cells by activation of the metabotropic ml muscarinic receptors that can generate intracellular heat production, and the intracellular heat production process can be imaged using thermal-sensitive fluorescence emission measurement by monitoring Eu-TTA phosphorescence intensity change as a function of time. Although the heat responses demonstrated in the reference are the result of low power heat generated by biochemical metabolism in the cells, the important lesson learned here and applied according to specific embodiments of the invention is that the intracellular heat production does occur in single cells. The non-uniformity of temperature distribution exists within and between cells and the heat propagation process sustains for a relative long period of time. The results are apparently contrary to the conclusion made by Rabin [19], which states that intracellular heating in microscopic scale is nearly impossible.

To effectively realize “intracellular cancer thermal-therapy”, the present invention uses nano-particle ferromagnetic resonance for intracellular cancer thermal ablation therapy and optionally also as internal thermometry.

3. Electron Spin Resonance for Imaging and Treatment

This invention utilizes the discovery that electron spin resonance can be used for effective and local heating of superparamagnetic particles, preferably superparamagnetic nanoparticles in, or adjacent to, biological specimens (e.g., cells, tissues, organs, organisms, etc.). The local heating obtainable using the methods described herein is effective in the hyperthermic (e.g., thermal ablation, temperature-induced apoptosis, etc.) treatment of cancers (or other conditions characterized by cellular hyperproliferation), the cosmetic ablation of tissues, and the like.

A high degree of specificity can be achieved using targeting of resonant frequency and selective update of the SPM or both of the two approaches.

The method of this invention are particularly well suited for therapeutic applications because they also permit visualization, preferably non-invasive visualization of the superparamagnetic particles and thereby of the cells, tissues, organs, etc. that the nanoparticles reside in. Visualization methods include, but are not limited to X-rays (the nanoparticles can act as contrast agents), magnetic resonance imaging (MRI), electron spin resonance imaging (eMRI), thermographic imaging (e.g., by detecting the signature of the heated nanoparticles), and the like. In various embodiments, the visualization can be performed simultaneously or independently of the particle heating.

Superparamagnetic particles are magnetic materials (e.g., ferromagnetic materials, ferromagnetic materials, etc.) with essentially zero magnetic coercively or spontaneous magnetization. At a zero applied magnetic field, the particles do not manifest magnetization and exert magnetic force on each other. In a non-zero magnetic field, due to long-range coupling of electron spins in the superparamagnetic materials, the spins align along the direction of the applied magnetic field. As a result, the spin population difference is nearly one below Curie temperature. Therefore, this approach provides the highest possible spin resonance absorption efficiency and can provide a significant and useful heating effect even at radio frequencies. At radio frequencies, the radiation can penetrate deep into a biological specimen, including human and animal, without heating up the other cells or tissues since these frequencies are far away from the water molecule absorption frequency spectrum.

A 3-Dimensional gradient configuration of magnetic field can be easily used to select specific locations that satisfy the equation (1) for spin resonance absorption heating. Importantly, superparamagnetic spin resonance imaging (with reduced RF frequency radiation) can be performed with the same equipment before, during, or after the heating therapy is performed. The required magnetic field is much lower (at least ten times) than that required for conventional MRI, making this technology relatively inexpensive (as compared to MRI).

Conventional NMR base MRI imaging can also be performed. In this case, the superparamagnetic particles serve as the relaxation T₂ contrast agent. Standard MRI equipment can be used here.

Since the nanoparticles are superparamagnetic, they do not exert magnetic force to each other and form clusters at zero magnetic field (Standley and Vaughan (1969) Electron Spin Relaxatin Phenomena in Solids, Plenum Press). This makes sample preparation and particle delivery very simple, as described elsewhere herein.

By applying, e.g., pulsed RF radiation power, the nanoparticles at the location that satisfies the equation (1) will be heated up to their Curie temperature. If the particle temperatures reach the Curie temperature, the particles lose their magnetic correlation and become paramagnetic. The spin population difference is then dramatically reduced and, as a result, the absorption power will go down. This effect gives the nanoparticles a convenient self-regulating mechanism to prevent over heating. Materials with a proper spin relaxation time constant (Poole (1983) Electron Spin Resonance (2nd Edition), A Wiley-Interscience Pub.) and Curie temperature can be chosen to form the nanoparticles to achieve optimized heating and therapeutic effects. Different sized nanoparticles can also be chosen to achieve the best delivery effect.

The present invention exploits existing biomedical and MRI technologies. For example, similar particles (e.g., oxides) have been used extensively as contrast agents in MRI applications. The existing MRI technologies and equipments can be readily borrowed for this technology.

A) Calculation of the Resonance Heating Effect with Superparamagnetic Particles

Due to the spin-lattice interaction of superparamagnetic particles, the absorbed microwave energy by spin resonance will be converted to thermal energy after precessing electron spins are relaxed. A simple calculation can be performed with YIG (Y₃Fe₅O₁₂) nanopowder as an example. YIG is a ferrimagnetic material having a net magnetization of 1400 emu/cm³ at room temperature (Goldman (1990) Modern Ferrite Technology, Van Nostrand Reinhold), or 1.5×10¹⁰ spins/μm³. Assuming the microwave frequency is 200 MHz, the relaxation time T₁ is 1 μs (Goldman (1990) Modern Ferrite Technology, Van Nostrand Reinhold; LeCraw and Spencer (1967) J. Phys. Soc. Jap. 17(Supplement B-I): 401), the microwave absorption power P for single spin is given by:

$$P = \frac{hf}{{T}_{2}} = 1.33 \times {10}^{- 19} W \cdot {spin}^{- 1} \vert$$

The power absorbed per unit volume is:
Pᵥₒₗᵤₘₑ=P×1.5×10¹⁰=2.0×10⁻⁹W·μm⁻³

If this energy is used to heat up the surrounding water with volume 10 times that of the YIG particle, using thermal capacitance of water of 4.2 Jcm⁻³C.° ⁻¹ and adiabatic conditions, the heating rate of the YIG-water region is given by:
R_{T}=Pᵥₒₗᵤₘₑ/4.2×10⁻¹²Jμm⁻³C.° ⁻¹×10=47.6C.° s⁻¹

This heating rate is rapid enough to kill cells in which the SPM nanoparticles are taken up. The above assumptions are considered conservative and more realistic conditions should give rise to more effective heating of the target(s).

B) Calculation of Input Microwave Power.

Calculations on the saturation power necessary to excite all spins of the nanoparticles in a selected area. To activate the spin resonance of ferromagnetic particles distributed in a large volume of human body, a relatively intense power is necessary. Using a coil as microwave radiator, and a one-dimensional gradient magnetic field is used to realize the computed tomography, the effective volume is the product of the cross section area and the linear dimension of the ferromagnetic particles. Assuming the particle size is 10 nm, the necessary power per unit area is
Pₐᵣₑₐ=Pᵥₒₗᵤₘₑ·10 nm=20W·m⁻²
Here simply assume that all the localized microwave power will be absorbed by the ferromagnetic particles to activate the spin resonance. Depending on the coil impendence, the input power to the coil can be calculated with the required Pₐᵣₑₐ. This level of power is very simple to realize in practical applications.
4. Ferromagnetic Resonance for Treatment and Imaging

In certain embodiments this invention also contemplates the use of nano-particle ferromagnetic resonance for localized tissue heating, ablation therapy (e.g. cancer therapy), and as internal thermometry. Ferromagnetic resonance occurs when the applied radiation field (microwave or RF) frequency matches the magnetic resonance frequency, which in general depends on magnetization and geometry of the magnetic particles as well as the applied magnetic field. The mechanism of ferromagnetic resonance is similar to that of NMR and ESR, but is much more powerful and versatile as a heating method. The theoretical basis for the resonance is discussed above.

As discussed above, because the energy difference between two Zeeman levels is small, at elevated temperature, thermal excitation causes spins to occupy both energy levels almost equally, leaving only a very small fraction of spins to contribute to the spin resonance. At room temperature and in a 5T magnetic field, this corresponds to a factor of 10⁻⁵ reduction in resonance absorption for a typical NMR. The same is true for paramagnetic electron spin resonance if the excitation frequency is the same as in NMR (necessary if radiation is going to penetrate human body). This explains why normal nuclear spin resonance (NMR) and electron paramagnetic resonance (EPR) absorptions are not effective in generating heat for therapeutic applications. In addition, nuclear spin resonance absorption heats up all protons, which is not suitable for targeted therapeutic treatment.

Very efficient heating, however, can be achieved by using ferromagnetic resonance absorption of, e.g., SPM nanoparticles. SPM particles are ferromagnetic materials with zero magnetic coercivity or spontaneous magnetization. In the absence of an applied magnetic field, the particles do not exhibit magnetization and there is no magnetic force between them. As a consequence, they do not interact magnetically with one another to clump together in the absence of an applied magnetic field. This ensures that the particles can be suspended uniformly in bio-compatible solutions and can readily be delivered to a particular location, e.g. in an organism before the RF magnetic field is applied.

In the presence of an applied magnetic field, however, the spins of unpaired electrons in these particles are correlated and the particles are magnetized. As a result, under the ferromagnetic resonance conditions, all unpaired electron spins are excited, rather than only 10⁻⁵ of total unpaired in NMR or ESR (the population difference).

Estimated heat absorption of magnetic nano-particles at ferromagnetic resonance is 10⁴-10⁶ times more efficient than that of Néel heating. This is because the Néel relaxation peak frequency for the hyperthermia technique is limited by particle-size related relaxation time typically around 10 KHz to 1 MHz, while the resonant frequency of FMR has no inherent limits and is practically determined by an externally applied static magnetic field. Even assuming uniform distribution of nano-particles, the temperature of the target (e.g. tumor region) can be raised by ˜10° C. within several seconds. In realistic non-uniform nano-particle distributions, “thermal-ablation” effects, e.g., where target (e.g., cancer) cells can be heated from 46° C. up to 56° C. can readily be realized. Therefore, this method can used to specifically kill targeted cells and/or tissues.

Since ferromagnetic resonance occurs only when the applied magnetic field and the electromagnetic radiation frequency satisfy the resonance conditions (similar to MRI imaging), heating can be directed to and limited to a specific a specific volume at a specific location. As a result, only cells ingested with nano-particles at a specific location will be heated and killed. In addition, the nano-particles can also serve as either conventional hydrogen NMR based MRI T₂ contrast agents, or electron spin resonance based MRI contrast agents, where the nano-particle spin resonance signal is used as contrast mechanism. As a consequence, image guided surgical heating therapy can be realized.

For ferromagnetic particle, the spin resonance occurs generally magnetization is saturated (M=Mₛ), i.e. when applied magnetic field exceeds a certain value to saturate the magnetization of the materials. As a result, the lower limit of ferromagnetic resonant frequency is related to Mₛ. The saturation magnetization is related to the composition of materials [22]. In order to control ferromagnetic resonance frequency under, e.g., about 0.5-1.0 GHz, which is sufficient to penetrate a body while remaining safe for that body, compositions that give rise to lower saturation magnetization values (and consequently lower ferromagnetic resonance frequency) have to be selected for nano-particle fabrication. Spin relaxation times (T₁ and T₂) can also be tuned in these materials to optimize the heating efficiency. Magnetic nanoparticles with surface chemical modifications have been used for various medical applications and therapeutic treatments [23-28]. Surface charge of SPM particles can be optimized to further enhance the differential up take ratio between cancer and normal cells [9].

5. Internal Thermometry and Image Guiding

Temperature Dependence of Electron Spin Resonance Frequency of Candidate Materials for Internal Thermometry Applications

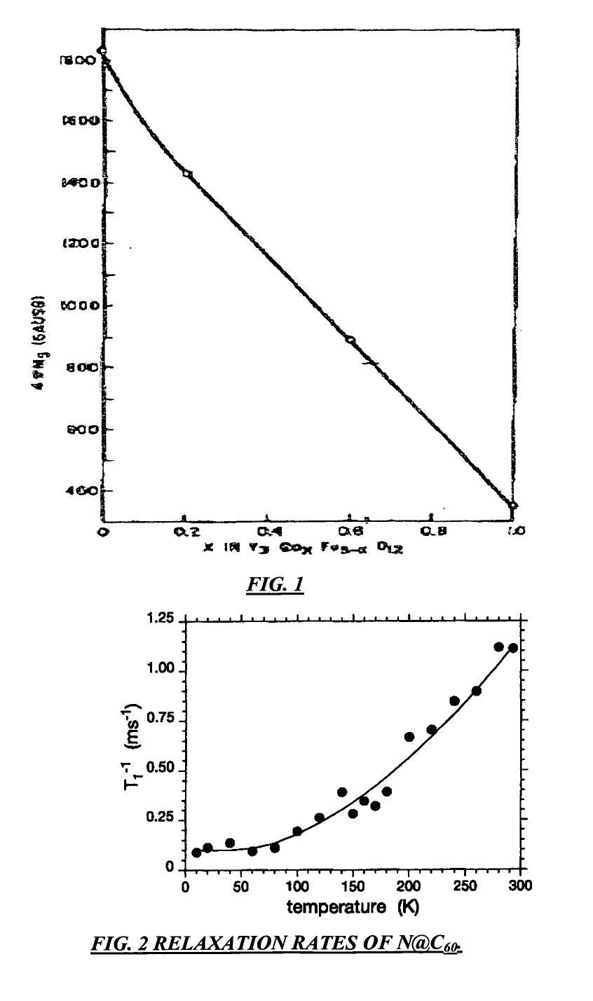

Furthermore, the ferromagnetic electron resonance frequencies of the nano-particles can be used as internal thermometry to monitor the temperature of the particle and cells because of the temperature dependence of ESR properties of these super-paramagnetic particles. One of the properties of ferri- or ferromagnetic materials is that the saturation magnetization depends on temperature [29]. Since the spin resonance frequency of ferri- or ferromagnetic materials is related to the saturation magnetization, by detecting the frequency change, the invention, according to specific embodiments can monitor the temperature in real time. FIG. 1 is a diagram illustrating room temperature saturation magnetization of Ga-YIG as a function of Ga concentration according to specific embodiments of the invention.

In addition, some paramagnetic materials, e.g. nitrogen endohedral fullerenes (N@C₆₀), have very long spin relaxation time of T₁ or T₂ that can be easily measured accurately and are strongly temperature dependent [30]. Detection of relaxation time is, therefore, also a very sensitive method to monitor temperature. In various implementations and embodiments, the invention characterized and employs the best materials for internal thermometry for particular applications. FIG. 2 is a diagram illustrating temperature dependence of the longitudinal relaxation rates of N@C60 according to specific embodiments of the invention.

If, rather than conventional NMR based MRI imaging facility (using SPM as T₂ contrast agents), ESR based imaging of SPM particles is implemented, then heat treatment, imaging and internal thermometry can be all accomplished with the same equipment at a much lower cost than conventional MRI since the required magnetic field for ESR is very low (<500 Gauss).

6. Instrument Set-up

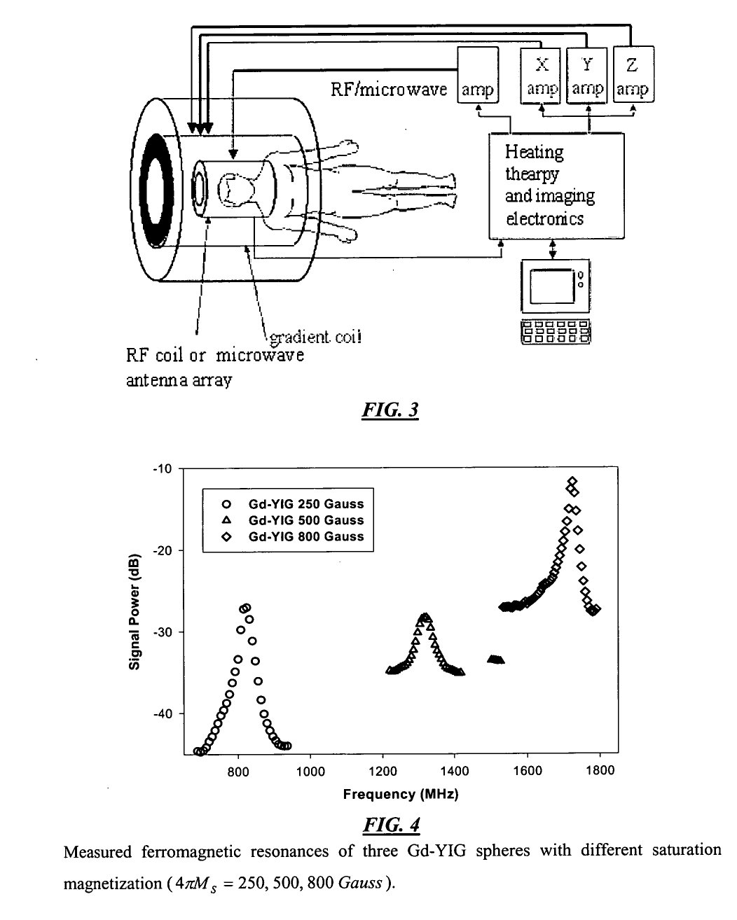

FIG. 3 illustrates an instrument set-up used for characterization of particle spin resonance detection and heating therapy. The setup is similar to the conventional MRI setup with the heating component integrated into it. Driven by the control electronics through X, Y, Z amplifier, the gradient coil can provide gradient magnetic field variable in 3 dimension which is necessary to localized the specific region of the tested sample or human body. The RF coil or alternatively the microwave antenna array is used as heating and spin resonance detection element. The 3D spin resonance imaging can be taken first with small microwave/RF power to locate the area where the heat therapy is necessary. Then the gradient field can be applied so that only the section contains the interesting region can satisfy the spin resonance condition. The heat therapy is processed then by adding higher power microwave through the RF coil to the whole body or microwave antenna array to a more focused region.

A typical ferromagnetic resonance of YIG sphere (diameter 0.3 mm) is shown in FIG. 4. The line width of the resonant peak shown in the resonance width is about 30 Oe, which is close to the reported value for YIG ceramic.

7. Spin Resonance Line Width and Heating Effect of Different Materials

A preliminary calculation outlining the heating capabilities of the YIG sphere was provided above. In certain embodiments, however, it is desirable to optimize several parameters: The relationship between the spin resonance line width and the power absorption rate and the heating efficiency; the particle size and its effect on the power absorption rate and the heating efficiency; and the best operating frequency.

The spin resonance line width is inversely proportional to the lifetime of the spin energy level. The broader the line width, the shorter the life time, which means the material may convert microwave energy into thermal energy more quickly. Therefore, higher levels of saturation power can be achieved. Broader line width materials, however, will decrease the microwave absorption efficiency when the RF source has a narrow bandwidth. For the purpose of selective excitation of the magnetic resonation and high spatial resolution in both treatment and imaging, the frequency bandwidth is desirably narrow. Therefore, optimized line width(s) are determined for the purpose of heating therapy and optionally simultaneous imaging.

8. Ferromagnetic Resonance Line Width and Heating Effects of Different Materials

Using calculations for the heating capabilities of the YIG sphere, it can further be determined how the spin resonance line width affects the power absorption rate and the heating efficiency and how the particle size affects the power absorption rate and the heating efficiency and therefore be determined what is the best operating frequency in particular embodiments. In specific embodiments, these determinations are made using a series of Ca(Gd)-doped YIG particles. [48].

In part, this is accomplished by measuring spin resonance signal amplitude curves as a function of input power to determine the saturation power level. The lifetime or line width can also be determined from the measurements. The two parameters determined there from are then compared with values from theoretical analysis. Selected materials with certain line width and saturation power level will be used for heating and temperature measurement.

The line width of the spin resonance can readily be detected using simple modifications to the set up shown in FIG. 3. To avoid absorption by water or biological fluids in the microwave region, the microwave frequency should be as low as possible since water absorption increase with the microwave frequency. On the other hand, the heating rate of magnetic resonance drops at lower frequency. In certain embodiments, the optimized frequency should be in the range of about 50 to about 2000 MHz, preferably about 100 to about 1000 MHz, more preferably from about 500 to about 1000 MHz. In this frequency range, an RF coil can be used as an RF transmitter and receiver. Compared to the resonator detector shown in FIG. 3, the RF coil may have a higher RF power transfer efficiency and can achieve uniform RF distribution in relatively larger regions. It also provides an open environment that is convenient to characterize the heating efficiency. Phase array antenna can be used here for radiation and detection of RF wave.

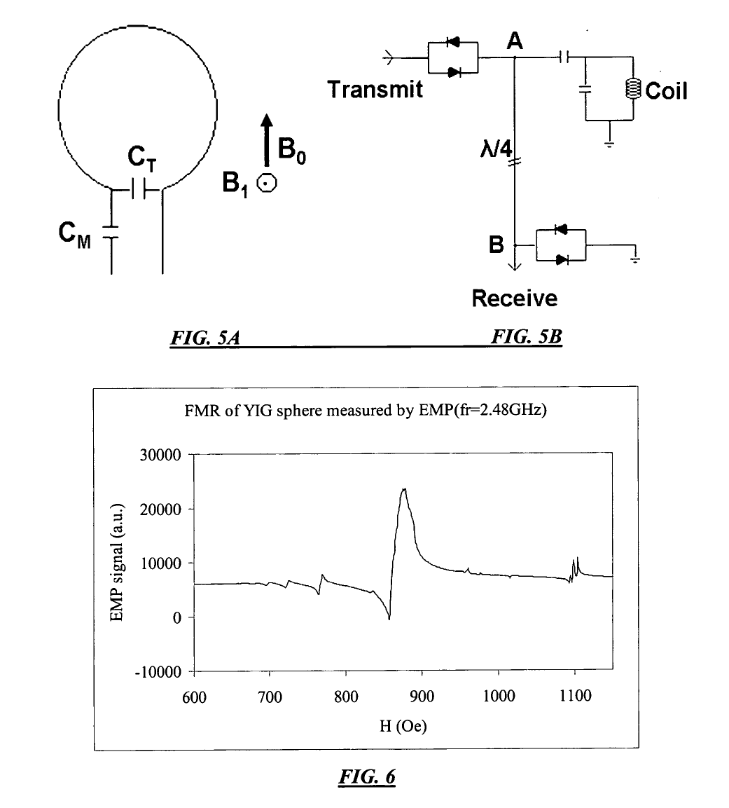

A surface coil can be applied to small volume for sample detection. In its simplest form it is a coil of wire coupled with a capacitor in parallel. The inductance of the coil and the capacitance form a resonant circuit, which is tuned to have the same resonant frequency as the spins to be detected. A second capacitor can be added in series with the coil, as shown in FIG. 5A, to match the coil impedance to, e.g., 50Ω. To prevent excitation pulse saturation or breakdown of the receiver electronics, which are designed to detect signals up to 6 orders lower than the input power, a simple protection circuit can be used as shown in FIG. 5B. To achieve a better signal noise ratio, the pulse RF signal can be used to replace the CW microwave signal. T his can be realized with the same microwave synthesizer by simply adding the pulse modulation control.

In certain embodiments, to study and characterize the heating effect of the particles, two methods can be used to measure the temperature increase cause by the spin resonance. In the first method, an infrared thermometer, e.g., with a temperature sensitivity of 1° C. can be used to monitor the radiation from the heated sphere (nanoparticles). Since it is impossible to focus the detection area as small as a nanoparticles due to the Abbe diffraction limit, a cluster of such powder, e.g., in a small glass tube can be used for the detection. In a second approach, a temperature sensitive paint (TSP) can be used to coat the nanoparticles. For example, a diluted layer of nanoparticles can be coated on a piece of glass slide. Then a thin layer of TSP can be coated. The heating effect is observed by the color change of the TSP. The temperature sensitivity of this method may be lower than the infrared thermometer, but it could directly monitor the surface temperature change of individual nanoparticles. When the sphere size smaller than 300 nm, the temperature change of individual nanoparticles will not able to be detected by this method. One can only estimate the temperature by the overall color change of the TSP coating covered on a cluster of spheres.

The relationship between heating up efficiency and nanoparticle size can also be empirically determined and optimized. Ideally, the nanoparticles size will not affect the heating efficiency if the heat generated by the RF absorption is only used to heat up the same volume. It is noted that there are several companies that produce commercially available superparamagnetic (e.g. ferrimagnetic, ferromagnetic, etc.) nanoparticles (e.g., Deltronic Inc). In certain embodiments, the nanoparticles range in size from about 1 nm to about 10 μm, preferably from about 10 nm to about 1 μm, more preferably from about 10 nm to about 100 nm.

9. Ferromagnetic Resonance (FMR)

Ferromagnetic resonance (FMR) is the electron spin resonance (ESR) in ferromagnetic or ferrimagnetic media. Due to long-range order of electron spins in ferro- or ferri-magnetic materials, the spin population difference is nearly one at room temperature. As a consequence, the sensitivity of FMR will not be reduced by the Boltzmann factor at room temperature for spin population difference, even if the radiation frequency is dramatically reduced. Therefore, this approach permits the use of radio frequency high FMR signals for heating and/or imaging in biological organisms and provides high heating efficiency. In certain embodiments, ferrimagnetic materials with narrow resonance line width are used.

10. Importance of Narrow Resonance Line Width

The importance of narrow resonance line width for high near-resonance sensitivity is seen in both the real and imaginary parts of the complex permeability μ=μ′+iμ″. In microwave (RF) circuits, μ′ controls the signal phase and μ″ controls the energy absorption or circuit Q factor. Their relations as a function of angular frequency ω can be expressed as:

$$\begin{matrix}\begin{matrix}{μ}^{′} = 1 + \frac{γ4π M ( ω - {ω}_{0} )}{( {ω}^{2} - {ω}_{0}^{2} ) + {{γ}^{2} ( Δ H )}^{2}} \\ \approx 1 + \frac{γ4π M ( ω - {ω}_{0} )}{{{γ}^{2} ( Δ H )}^{2}} ( near resonance )\end{matrix} & 3 A \\ and \\ & \\ \begin{matrix}{μ}^{″} = \frac{γ4π M γ ( Δ H )}{( {ω}^{2} - {ω}_{0}^{2} ) + {γ ( Δ H )}^{2}} \\ \approx \frac{4 π M}{Δ H} ( at resonance ) ,\end{matrix} & 3 B\end{matrix}$$

where 4πM is the magnetization comprising the volume density of individual magnetic moments m, ω₀ is the resonance frequency, and ΔH is the line width. The factor γ is the gyromagnetic constant and is derived from the Larmor precession relation between frequency and field, given by:

$${ω}_{0} = γ H = \frac{g \vert e \vert}{2 mc} H , \qquad ( 4 )$$

where e is the electron or proton charge, m is the particle mass and c is the velocity of light, and g (˜2 for spins) is the spectroscopic splitting factor. Note that e is the same magnitude for both protons and electrons, but mₙ for protons is greater than mₑ for electrons by a factor 1836, thereby reducing the resonance frequency by a factor of more than 10³ for a given magnetic field intensity H.

From equation 3B, the imaginary part of susceptibility u″ is proportional to 1/ΔH (ΔH is line width), and μ″ is directly related to the RF energy absorption of the material, which means that materials with narrow spin resonance line width will have high RF absorption efficiency and can be easily heated for a given single excitation frequency coincide with the spin resonance frequency.

In certain embodiments, the RF will range from about 400 MHz to about 1 GHz to heat the material. In this context, a typical/reasonable pulse width is about 1 μs, which corresponds to a line width of 1 MHz and quality factor of about 500˜1000. If the line width of selected material is too broad (low quality factor), the absorption band of the material will not be covered effectively by the RF pulse spectrum, which will also decrease the heating efficiency. Thus the spin resonance quality factor of the selected material should be larger than 10, more preferably larger than about 50, still more preferably larger than about 100, 200, or 500. In certain embodiments, the spin resonance quality factor (O) ranges from these values up to about 800, 1000, 15000, 2000, or 3000. In certain embodiments, the Q factor ranges from about 100 to about 1000.

Several factors contribute to the line width, chief among which are (1) spin-lattice interactions of individual spins, characterized by a relaxation time τ₁, and (2) incoherent precession phasing of spins, characterized by a relaxation time τ₂ that arises from misaligned spins coupled by dipolar interactions. Precession phase decoherence can also occur in exchange ordered electron spin systems by spin wave generation, particularly in higher power cases where crystal imperfections or non-uniform RF fields exist in a specimen having dimensions greater than the wavelength of the RF signal. These mechanisms are generally considered to be homogeneous and produce a Lorentzian line shape.

Inhomogeneities can cause severe broadening by creating local regions of different resonance frequencies in a Gaussian-type distribution. Most common among these cases are polycrystalline ferromagnetic specimens with crystal grains of random crystallographic orientation with varying magnetic anisotropy bias fields and structural inhomogeneities such as nonmagnetic phases, porosity and grain boundaries that can broaden the effective ΔH of a typical ferrite by more than a hundred oersteds. In small specimens with rough surfaces, demagnetization effects on line width, similar to those of bulk porosity, have been observed. For this reason, the discussion of FMR that follows focuses primarily on relatively polished single crystal specimens where only the homogeneous broadening effects from the relaxation rates τ₁⁻¹ and τ₂⁻¹.

For homogeneous relaxation broadening ΔH=(γτ)⁻¹ (5), where the relaxation time τ can be a resultant of both τ₁ and τ₂ contributions, but is generally dominated by only one of them. Relaxation rates of paramagnetic systems are influenced primarily by τ₂⁻¹, with the possible exception of certain electron cases where fast relaxing ions allow two-phonon Raman processes to render τ₁⁻¹ large enough to approach or exceed τ₂⁻¹. With ferromagnetic specimens, the spin-spin relaxation rate in ideal situations is effectively zero because of complete spin alignment means perfect precession phase coherence. Although τ₁⁻¹ becomes the dominant relaxation parameter, only selected ions can fulfill the goal of narrow line width. Estimated values of these parameters are listed in Table 1 for typical situations.

TABLE 1
Estimates of gyromagnetic resonance parameters at T = 300° K.

| | 4πM | τ₁ | τ₂ | ΔH | μ″ |
|---|---|---|---|---|---|
| | (G) | (sec) | (sec) | (Oe) | — |
| NMR | ˜2 | >10⁴ | ˜10⁻⁴ | ˜0.05 | ˜40 |
| | (conc.) | | | | |
| EPR (Fe³⁺) | 20 | >10⁻⁶ | ˜10⁻⁷ | ˜5 | ˜4 |
| | (dilute) | | | | |
| | 2000 | | ˜10⁻⁹ | ˜500 | ˜4 |
| | (conc.) | | | | |
| FMR (Fe³⁺) | 2000 | >10⁻⁶ | → ∞ | <0.5 | 4000 |
| | (conc.) | | | | |

The resonance frequency ω₀ can vary with orientation of the specimen in different ways. For paramagnets, γ can be sensitive to crystallographic direction, and in some case, range widely. However, γ is relatively isotropic in ferrimagnets with d⁵ or d⁷ magnetic ions. The main sources of anisotropy come from surface poles that induce demagnetizing fields proportional to 4πM inside the specimen, and from fields proportional to ratio of the magnetocrystalline anisotropy fields that are associated with specific crystallographic axes.

For a fully magnetized ellipsoidal specimen with H and M aligned with the z-axis, the resonance frequency is expressed as
ω₀=γHᵢ, 6
where the internal field for resonance is given by

$${H}_{r} = {\{ \begin{matrix}[ H + ( {H}_{Kx} - {H}_{Kz} ) + ( {N}_{Dx} - {N}_{Dz} ) 4 π M ] \times \\ [ H + ( {H}_{Ky} - {H}_{Kz} ) + ( {N}_{Dy} - {N}_{Dz} ) 4 π M ]\end{matrix} \}}^{\frac{1}{2}} \qquad 7$$

The subscripts x and y refer to the two axes of the ellipsoid that are orthogonal to the z direction of H in the coordinate system selected. Note that Hᵢ reduces to the applied field H when all of the demagnetizing factors are zero.

For resonance to occur with H along the z-axis, H_{rf} must have a component in the xy-plane, but values of the H_{K} anisotropy fields and the N_{D} factors will be sensitive to the direction of H within the plane. Applied to the limiting case of a thin flat plate with N_{Dx}=1, and N_{Dy}, N_{Dz}=0, and H_{K} terms ignored, equation 6 simplifies to:

$$\begin{matrix}{ω}_{0} = {γ [ H ( H + 4 π M ) ]}^{\frac{1}{2}} & ( H in plane ) \\ {ω}_{0} = γ ( H - 4 π M ) & ( H normal to plane )\end{matrix} \qquad 8$$

For a long slender cylinder (acicular particle) aligned with the z-axis, N_{Dx}, N_{Dy}=½, and N_{Dz}=0. The resonance frequency is then:

$$\begin{matrix}{ω}_{ 0} = γ ( H + 2 π M ) & ( H parrallel to long axis ) \\ {ω}_{ 0} = {γ [ H ( H - 2 π M ) ]}^{ \frac{1}{ 2}} & ( H normal to long axis )\end{matrix} \qquad 9$$

For a sphere, N_{Dx}=N_{Dy}=N_{Dz}=⅓, and the shape demagnetizing factors cancel, so that Hᵢ=H.
ω₀=γH 10

As a consequence, care is taken in selecting specimen shapes. From equation 10, it is clear that spherical particles are most suitable for this purpose. In addition, dispersal of the individual ferrimagnets is also important to avoid dipolar interactions on a macroscopic scale, e.g., super-paramagnets.

11. Quantitative Heating Efficiency Comparison between FMR heating and Néel Heating

A quantitative theoretical analysis of both Néel heating and ferromagnetic resonance heating according to specific embodiments of the invention shows the effectiveness of the later approach. Among several heating methods in hyperthermia technology, Néel heating is currently the most effective way when the particle size is smaller than the single magnetic domain size, i.e. the particles are so called super-paramagnetic particles. Néel heating works at the Néel relaxation frequency, which results from the thermal activation of re-orientation of particle magnetization polarized by the external alternating magnetic field. In the case of FMR, the absorbed RF energy is transferred to thermal energy during the relaxation of the resonant precessing spin. Since both FMR and Néel heating are realized through magnetic relaxation, their heating efficiency can be evaluated using their imaginary magnetic susceptibility χ″ [21]:

$$P = \frac{1}{2} {χ}^{″} ω {H}_{1}^{2} , \qquad ( 3 )$$

where P is the RF energy absorption rate per unit volume, ω and H₁ are the frequency and the magnetic field magnitude of the RF field, respectively. The Néel relaxation and ferromagnetic spin resonance can be described in a unified solution of the modified Bloch equation [36-38]:

$${χ}^{″} = \frac{1}{2} {χ}_{0} ωτ \frac{1}{1 + {( ω - {ω}_{0} )}^{2} {τ}^{2} + {γ}^{2} {H}_{1}^{2} {τ}^{2}} \qquad ( 4 )$$

Where χ₀ is material's static susceptibility, ω₀ is magnetic spin resonance frequency, γ and τ are gyromagnetic ratio and relaxation time respectively. When ω=ω₀, magnetic spin resonance occurs, and χ″ becomes (assuming saturation term γ²H₁²τ²<<1):

$${χ}^{″} = \frac{1}{2} {χ}_{0} {ω}_{0} τ . \qquad ( 5 )$$

However, when ω₀ approaches zero, i.e. no applied DC magnetic field, Eq.(4) becomes the case of Néel relaxation (also assuming the saturation term γ²H₁²τ²<<1):

$${χ}^{′′} = \frac{1}{2} {χ}_{0} ω τ \frac{1}{1 + {ω}^{2} {τ}^{2}} . \qquad ( 6 )$$

The Néel relaxation peak takes place at ωτ=1.

Comparing Eq.(5) with Eq.(6), one can easily find that the biggest difference in the imaginary susceptibilities of FMR and Néel relaxation is the difference in operating frequencies. It is obvious that the Néel relaxation peak frequency for the hyperthermia technique is limited by particle-size related relaxation time due to ωτ=1, and therefore is around 10 KHz to 1 MHz for typically τ=10⁻⁴-10⁻⁶ second.

On the other hand, the resonant frequency of FMR has no inherent limits. It is practically determined by an externally applied static magnetic field. For hyperthermia cancer treatment purpose, in specific embodiments, the present invention uses a resonant frequency of around 0.5˜1 GHz, which is safe while penetrable to human body. In other words, the spin resonance frequency of FMR in FMR heating therapy according to specific embodiments of the invention is at least two orders of magnitude higher than the Néel relaxation peak frequency. Since the energy absorption P is proportional to ω² according to Eq.(3) that converts the heating efficiency of FMR to be at least 10⁴-10⁶ times higher than that of the Néel heating hyperthermia technique.

12. Estimation of the Resonance Heating Effect with SPM Particles

A further analysis calculates the heating from ferromagnetic resonance of SPM nano-particles and the heat transfer process from the nano-particle to its surrounding environment. The analysis shows that the SPM nano-particle power absorption per unit volume at the spin resonance frequency ω₀ is

$$P = \frac{γ {M}_{0} {ω}_{0} {T}_{2} {H}_{1}^{2}}{1 + {T}_{1} {T}_{2} {γ}^{2} {H}_{1}^{2}} . \qquad ( 7 )$$

Under the saturation conditions (T₁T₂γ²H₁²>>1), Eq.(7) becomes

$$P = \frac{{M}_{0} {ω}_{0}}{{T}_{1} γ} . \qquad ( 8 )$$

Using typical values for Ga or Ca doped YIG particles, T₁=T₂=10 ns (note, this value is far higher than most commonly used materials, which have T₁<1 ns—in other words, the estimation here is the most conservative estimation) 4πM₀=250 Gauss, ω₀=2π×500 MHz, results yield: P=3.6×10¹⁰ W/m³.(9)

Rudolf Hergt built a model for Néel relaxation and obtained a power absorption equation in his paper [39] similar to Eqs.(3) and (6) in our Néel relaxation analysis. Unfortunately, the maximum power absorption of over 10⁹ W/m³ under conditions of ω=2×10⁶ s⁻¹ and AC magnetic field amplitude of 6.5 kA/m he derived was an erratic mistake because Hergt's equation is valid only for small fields approximation (mH<<kT). The very large magnetic field (mH>>kT) used in his calculation renders a huge overestimated power absorption value. If a small field parameter (˜100 A/m) is used, the power absorption we obtained is only 8×10⁵ W/m³. Compared with Eq.(9), the Néel relaxation heating power is 10⁴ to 10⁵ times lower than the FMR heating power. This heating difference is consistent with the theoretic analysis above.

Assuming the power in Eq.(9) is used to heat up a surrounding amount of water with 30 times the volume of the YIG particles under adiabatic conditions, the heating rate is (considering the specific heat of water of 4.185 J/cm³° C.): T_{R}=286° C./s. (10)

The result suggests that only 0.03 second is needed to increase temperature by 10° C. (at which cells start to die) with a typical cell size of 5 μm and the maximum uptake of SPM particles of 5×10⁻¹⁰ g. In this calculation, assume the SPM particles inside the cancer cell that are surrounded by a layer of thermal insulating membrane.

Even if the cancer cell membrane is assumed to be as thermally conductive as water, using Rabin's macro-scale model [19], the temperature increase in a particle uniformly distributed region under steady state is

$${θ}_{t} = \frac{{pD}_{t}^{2}}{8 k} , \qquad ( 11 )$$

where p is average power absorption per unit volume in the treated region, Dₜ is the diameter of the region, and k is water thermal conductivity 0.64 W/m° C. Assuming the nano-particle volume concentration is 1/30 [19], to get 10° C. temperature increase using the heating power in Eq.(9), the minimum treatable region (the smallest volume can be heated in a steady of uniform particle distribution) is only 0.2 mm.

Facilities to measure spin resonance and test heating effect of different materials have been developed by Internatix and include a Microwave Electron Spin Resonance Detection system with an electromagnet up to 10 kOe. The ferromagnetic resonances of different Ga-doped YIG spheres (diameter 0.3 mm) have thus been successfully measured. Shown in FIG. 6 are the measured results.

13. Instrument Design of the Gradient Magnetic Field for Imaging Assisted, Differential Heating Therapy for Cancer

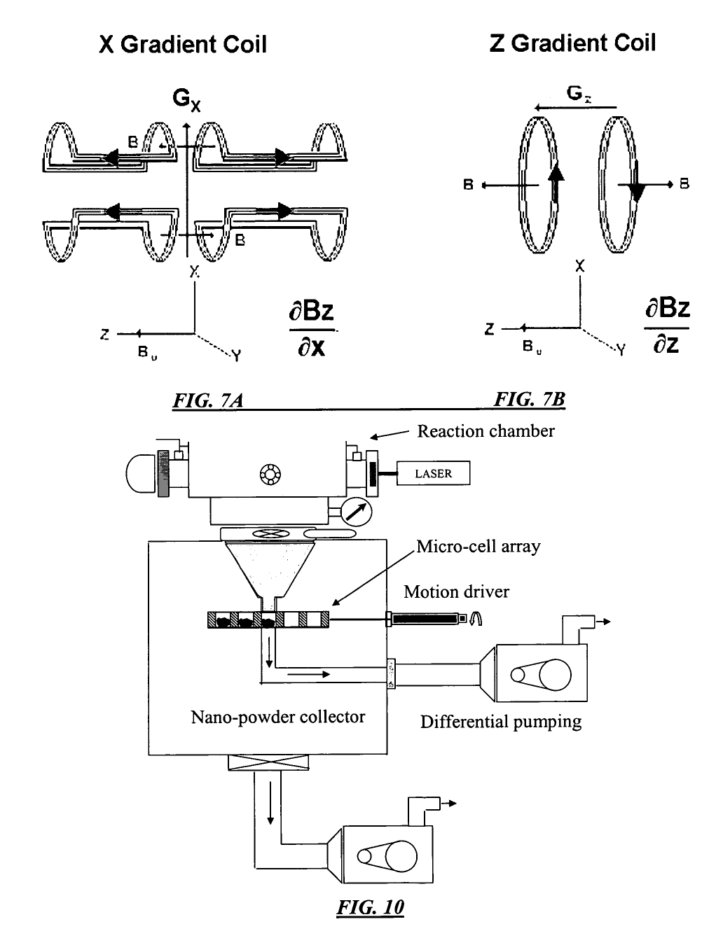

For 3D heating capabilities with larger specimens (organisms), the surface coil is preferably replaced with a commercial available birdcage coil, which can provide uniform RF distribution in bigger volume. To realize localized heating and spatially resolved imaging, a magnetic field gradient is provided. FIGS. 7A and B illustrate the generation of gradient field for 3-D heating and/or imaging.

Magnetic field gradients are spatially dependent variations in the magnetic field created by electrical DC currents in specifically designed coil arrangements. For example, a linear magnetic field gradient that varies spatially along the z direction of the main magnet can be produced using a Maxwell pair of coils as pictured in FIG. 7B. Such a magnetic field, when applied to a sample of homogeneous material like water, causes the spins on one side of the sample with respect to the z direction to have a different frequency from spins on the other side of the sample. A distribution of frequencies will be obtained along the sample. The amount of magnetization at each frequency will be the integral of the signal along a surface perpendicular to the applied field gradient. An x gradient is obtained using a coil configuration as shown in FIG. 7A, and need only be rotated by 90 degrees to obtain any gradient. Both of these make fields that add or subtract from the main magnetic field pointing along z but the magnetic field strength varies in the x or y direction.

The 3D heating and imaging setup preferably controls the gradient field and RF pulse in a specific time sequence. Software controlling the device can offer the following functions: 1) Control of the gradient field to realize the planar selection for heating and magnetic resonance detection; 2) Control of the RF pulse sequence according to the applications. In certain embodiments, for heating, a continuous 180° pulse is provided with period related to the relaxation time of the magnetic resonance. For imaging, in certain embodiments, a 90° pulse is provided to observe the relaxation signal. 3) The FFT functions can be used to analyze the line width of the spin resonance (Ernst et al. (1987) Principles of Nuclear Magnetic Resonance in One and Two Dimensions, Clarendon Press Oxford) and reconstruct the image when phase encoding and frequency encoding pulse is used to realize the magnetic resonance imaging.

14. Superparamagnetic Material Selection.

Compared to NMR, electron paramagnet resonance offers larger individual magnetic moments, but has broader associated line widths resulting from relaxation times that are shortened by spin-orbit coupling in all cases except the half-filled d shell ions, i.e., 3d⁵ of Fe³⁺, Mn²⁺ or rare earth 4f⁷ of Gd³⁺, Eu²⁺. Strong dipolar coupling also reduces τ₂ when concentrations of paramagnetic centers are increased in attempts to raise the dc susceptibility.

For selection of particles, single-crystal ferrimagnetic spheres offer the advantages of high detectability through large magnetizations and narrow FMR lines. For example, yttrium-iron garnet Y₃Fe₅O₁₂ and γ-Fe₂O₃ are two well-known materials suitable for this application. Different dopants can be added to lower the spin resonance frequencies of these materials for medical applications. Magnetic garnets and spinels are also chemically inert and indestructible under normal environmental conditions.

An illustrative list of potential dilutant ions for the generic {c}₃(a)₂[d]₃O₁₂ and spinel A[B]₂O₄ ferrite compounds that preserve produce different ω₀ values while preserving the narrow ΔH requirement is presented in Table 2. Among those ions to be preferably avoided are those with fast spin-lattice relaxation rates, specifically members of the 3d or 4f transition series without half-filled shells, particularly Co²⁺, Fe²⁺, or any of the lanthanide (rare earth) series not listed in Table 2.

TABLE 2
Ferrite dilution ions for preserving narrow line width.

| | |
|---|---|
| Garnet {c}₃(a)₂[d]₃O₁₂ | Spinel A[B]₂O₄ |

| {c} | (a) octahedral | A | [B] |
|---|---|---|---|
| dodecahedral | [d] tetrahedral | tetrahedral | octahedral |
| Y³⁺ (highest | Fe³⁺ | Fe³⁺ | Fe³⁺ |
| purity) | | | |
| La³⁺ (highest | Mn²⁺ | Mn²⁺ | Mn²⁺ |
| purity) | | | |
| Gd³⁺ (highest | Ru³⁺ | Ru³⁺ | Ru³⁺ |
| purity) | | | |
| Eu²⁺ (highest | Cu¹⁺ | Cu¹⁺ | Cu¹⁺ |
| purity) | | | |
| Na¹⁺ | V³⁺ [d], Ni²⁺ (a) | V³⁺ | Ni²⁺ |
| K¹⁺ | Cr⁴⁺ [d], Cu³⁺ (a) | Cr⁴⁺ | Cu³⁺ |
| Rb¹⁺ | Mo⁴⁺ [d], Cr³⁺ (a) | Mo⁴⁺ | Cr³⁺ |
| Tl¹⁺ | W⁴⁺ [d], Mo³⁺ (a) | W⁴⁺ | Mo³⁺ |
| Ag¹⁺ | Nb³⁺ [d], W³⁺ (a) | Nb³⁺ | W³⁺ |
| Au¹⁺ | Zn²⁺ | Zn²⁺ | Zn²⁺ |
| Hg¹⁺ | Mg²⁺ | Mg²⁺ | Mg²⁺ |
| Ca²⁺ | Al³⁺ | Al³⁺ | Al³⁺ |
| Sr²⁺ | Ga³⁺ | Ga³⁺ | Ga³⁺ |
| Ba²⁺ | In³⁺ | In³⁺ | In³⁺ |
| Hg²⁺ | Sc³⁺ | Sc³⁺ | Sc³⁺ |
| Pb²⁺ | Ti⁴⁺ | Ti⁴⁺ | Ti⁴⁺ |
| Bi³⁺ | Zr⁴⁺ | Zr⁴⁺ | Zr⁴⁺ |
| In³⁺ | Hf⁴⁺ | Hf⁴⁺ | Hf⁴⁺ |
| Sc³⁺ | Si⁴⁺ | Si⁴⁺ | Si⁴⁺ |
| | Ge⁴⁺ | Ge⁴⁺ | Ge⁴⁺ |
| | Sn⁴⁺ | Sn⁴⁺ | Sn⁴⁺ |
| | V⁵⁺ | V⁵⁺ | V⁵⁺ |
| | Nb⁵⁺ | Nb⁵⁺ | Nb⁵⁺ |
| | Ta⁵⁺ | Ta⁵⁺ | Ta⁵⁺ |
| | P⁵⁺ | P⁵⁺ | P⁵⁺ |
| | As⁵⁺ | As⁵⁺ | As⁵⁺ |
| | Sb⁵⁺ | Sb⁵⁺ | Sb⁵⁺ |

15. Surface Modification and Suspension of Magnetic Nanoparticles in Bio-Compatible Solutions

Applications of ferrofluids of magnetite particles coated with aminosilane for hyperthermia cancer therapy have been reported by Jordan and coworkers [9, 13, 40]. The basic science for silane coating was developed by Arkles back to 1977 [41]. Extending the chemistry developed by B. Arkles, the present invention in specific embodiments involves coating procedures to make surface modification of magnetic nano-particles for bio-compatible solutions.

An Example Procedure

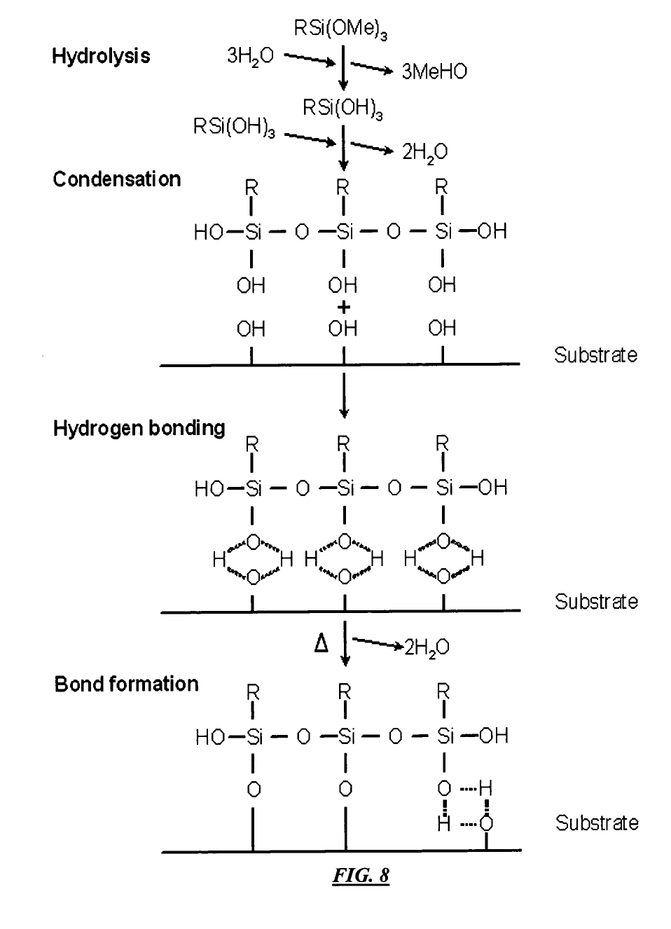

One example procedure for surface modification and suspension of magnetic nanoparticles in bio-compatible solutions according to specific embodiments of the invention is shown in FIG. 8. As illustrated in FIG. 8, (1) the first step is the hydrolysis of the three labile groups of (MeO)₃SiCH₂CH₂CH₂NH₂. 2% w/v of (MeO)₃SiCH₂CH₂CH₂NH₂ is to be dissolved in de-ionized water under ultrasonic mixing conditions for several minutes to formulate (OH)₃SiCH2CH2CH2NH2. (2) The second step is the condensation of (OH)₃SiCH₂CH₂CH₂NH₂ to formulate oligomers as follows: (OH)₂Si(R)—O—(R)Si(OH)—O-Si(R)(OH)₂, R═CH₂CH₂CH₂NH₂ via ultrasonic mixing for another five to ten minutes.

(3) The third step is to uses a pre-prepared colloidal nano-particle (YIG) solution without aggregates. The colloidal solution's pH is adjusted by ammonium hydroxide to keep the pH at 8-9 to ensure that OH groups are surrounding the nano-particles. The YIG nano-particle colloidal solution is to be mixed with the Si oligomers solution under ultrasonic to form hydrogen bonds between the OH groups of nano-particles and of the Si oligomers.

(4) In the fourth step, a covalent linkage will be formed with the substrate by loss of water to form Fe—O—Si bonds under ultrasonic mixing and at temperatures around 60° C. The fifth step is to isolate a solution of nano-particles (YIG) with aminosilane shells from uncoated polymers and MeOH through gel filtration chromatography.

YIG with a Dextran Type Shell

A second approach according to specific embodiments of the invention to make surface modified magnetic nano-particles bio-compatible is to prepare YIG with a Dextran type shell. The procedures of making YIG—dextran particles have been established according to specific embodiments of the invention based on the reports published in literature [42-46]. Magnetic YIG—dextran particles are prepared by suspending YIG nanoparticles first in de-ionized water. The solution is mixed ultrasonically along with pH adjustment by adding acetic acid or ammonium hydroxide depending upon charge type required for the specific applications.

An equal volume of a 20% (w/v) dextran (40 kDa) solution in deionized water is to be mixed with the YIG solution and kept at a constant temperature slightly above room temperature (˜35° C.) for a certain period of time (˜15 minutes) under ultrasonic mixing to let the coating occur.

The YIG—dextran particles are to be separated from unbound dextran by gel filtration chromatography on Sephacryl S-300. The reaction mixture is to be eluted with buffer containing sodium acetate and NaCl at pH 6.5. The purified YIG—dextran particles collected in the void volume are expected to have a concentration of 7-10 mg/ml. The coating improves dispersibility, chemical stability and reduces toxicity [47].

16. Synthesis of Nano-particle Using Laser Pyrolysis

In specific embodiments one or more types of nanoparticles of use as described herein is synthesized using proprietary combinatorial laser pyrolysis (CLP) systems described in co-assigned patent applications. Such nano-particles may have different chemical compositions and particle sizes to meet the requirement of FMR nano-particle thermal ablation cancer therapy as described herein. [31-34].

The combinatorial laser pyrolysis (CLP) system is one of the proprietary combinatorial materials synthesis techniques that has been proven to be unique and powerful in the high throughput synthesis of nano-particles. The laser pyrolysis technique was established as an alternative approach to synthesize nano-particles with the advantages over other chemical synthesis approaches in work by Canno [35]. The advantage are a) the small particle size, (b) the narrow particle size distribution, and (c) the nearly absence of aggregation. By implementing the combinatorial material synthesis capability, the CLP system enables us to develop various nano-particles with different chemical compositions and nano-sizes meeting different requirements of variety of applications.

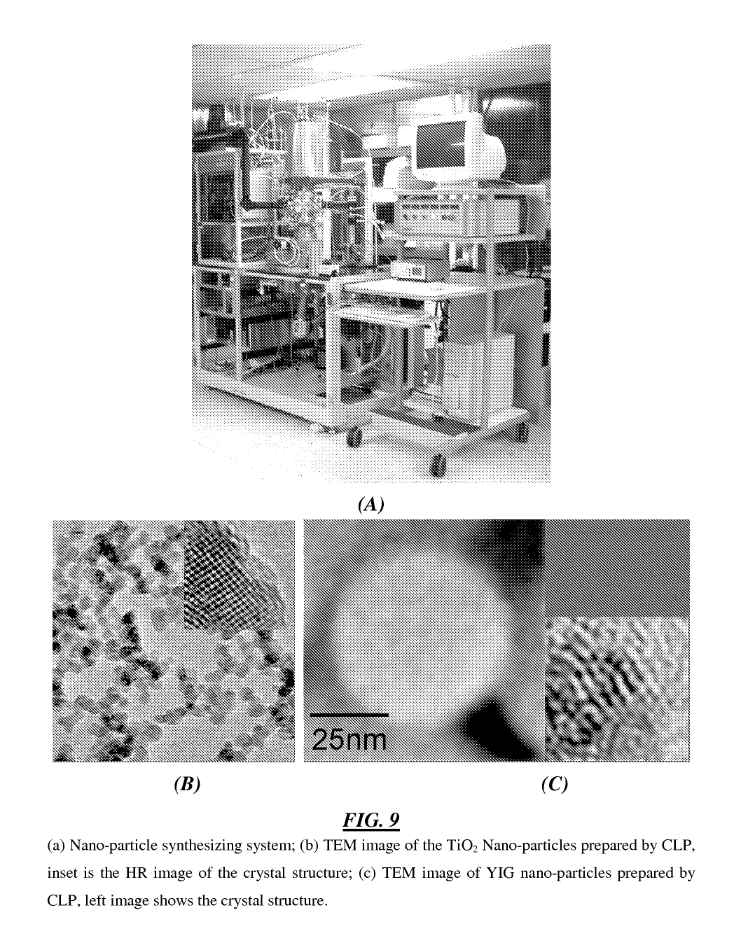

FIG. 9(a) shows the system. A CO₂ laser is used to heat gas molecules delivered by a multi-precursor ink-jet chemical vapor delivery system. An advantage of using a laser is its narrow spectral width, which allows efficient coupling between the light and molecular precursors that have exact wavelength of absorption (over 15% of laser power consumed). The CLP system consists of two independently controlled source injectors to deliver organometallic precursors for the metal elements of desired chemical compositions. The injection rate and volume of two injectors are precisely controlled by a computerized controller; this allows our combinatorial approach of powder production: systematically varying the ratio between metal (I) and metal (II), as well as the dopant density. The vaporized precursors mixed with carrier gas and heat adsorption gases are heated by the laser beam in reaction chamber forming a flow of nano-powders. O₂ or air is introduced into the reaction chamber for the synthesis of oxides. The air-sensitive particles can also be synthesized as the system is vacuumed with background pressure of ˜1×10⁻⁶ Torr. The nano-powders follow the gas downstream along the pumping direction, and are collected by means of micro-cell array with differential pumping (as illustrated in FIG. 10). With the motion control, each cell collects the discrete nano-particle samples with different chemical composition or synthesized under different experimental conditions (such as gas flow, vacuum pressure), which leads to different size of particles. The structure and size of powders are subsequently characterized using transmission electron microscopy (TEM) and X-ray diffraction spectroscopy.

FIG. 9(b) and FIG. 9(c) shows the SEM and TEM images of TiO₂ and YIG nano-particles respectively synthesized using this system. Although the synthesis conditions are still under development, the crystal structure of nano-particle (illustrated by TEM images) and shape are useful for many applications. In this particular synthesizing process, Argon was used as the carrier gas and C₂H₄ as absorbing gas. Their flow rates were controlled independently by the mass flow controller. The CW CO₂ laser power used to heat up the gas molecular precursors through absorbing gas to form nano-particles ranged from 100 W to 250 W with the beam size of 5 mm. The chamber was first pumped to below mTorr range then the gases were introduced into the chamber for reaction process, which results in the rising of pressure of reaction chamber to 10-100 Torr. The reaction was initiated by turning on laser beam. This unique capability enables us to quickly optimize composition and size of nano-particles for heating efficiency.

In further embodiments, the magnetic moment and particle size of the YIG nano-particles is tailored and fine-tuned to optimize the function of the YIG in the cancer treatment. The YIG nano-particles with different doping densities of Ca(Gd) and different nanometer-sizes will be synthesized using CLP. The C₆H₈Fe(CO)₃ and Y(OC₄H₉)₃ are used as precursors for Fe and Y respectively. Ca(THD)₂ (THD=2,2,6,6-tetramethylheptanedionate) and Gd(TMHD)₃ (TMHD=2,2,6,6-tetramethylheptane-3,5-Dionate) are the precursors for Ca and Gd respectively. The organometallic precursors will be dissolved into hexane and delivered into the reaction chamber through the CVD injectors. The particle size can be controlled through varying the experimental conditions, such as flow of precursor, the pressure of reaction chamber.

17. Pharmaceutical Compositions

The superparamagnetic nanoparticles or nanoparticles can be useful for parenteral, topical, oral, or local administration (e.g. injected into a tumor site), aerosol administration, or transdermal administration, for prophylactic, but principally for therapeutic treatment. The pharmaceutical compositions can be administered in a variety of unit dosage forms depending upon the method of administration. For example, unit dosage forms suitable for oral administration include powder, tablets, pills, capsules and lozenges. It is recognized pharmaceutical compositions of this invention, when administered orally, can be protected from digestion. This is typically accomplished either by complexing the active component with a composition to render it resistant to acidic and enzymatic hydrolysis or by packaging the active ingredient(s) in an appropriately resistant carrier such as a liposome. Means of protecting components from digestion are well known in the art.

The pharmaceutical compositions of this invention are particularly useful for parenteral administration, such as intravenous administration or administration into a body cavity or lumen of an organ. The compositions for administration will commonly comprise a solution of the nanoparticles and/or nanoparticles treated for intercellular update dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of chimeric molecule in these formulations can vary widely, and will be selected primarily based on fluid volumes, viscosities, body weight and the like in accordance with the particular mode of administration selected and the patient's needs.

The compositions containing the nanoparticles or a cocktail thereof (i.e., with other therapeutics) can be administered for therapeutic treatments. In therapeutic applications, compositions are administered to a patient suffering from a disease, e.g., a cancer, in an amount sufficient to cure or at least partially arrest the disease and its complications when appropriately utilized with electron spin resonance to effect heating of the nanoparticles. An amount adequate to accomplish this is defined as a “therapeutically effective dose.” Amounts effective for this use will depend upon the nature of the disease and the general state of the patient's health.

Single or multiple administrations of the compositions may be administered depending on the dosage and frequency as required and tolerated by the patient. In any event, the composition should provide a sufficient quantity of the compositions of this invention to effectively treat the patient.

It will be appreciated by one of skill in the art that there are some regions that are not heavily vascularized or that are protected by cells joined by tight junctions and/or active transport mechanisms that reduce or prevent the entry of macromolecules present in the blood stream

One of skill in the art will appreciate that in these instances, the therapeutic compositions of this invention can be administered directly to the tumor site. Thus, for example, brain tumors can be treated by administering the therapeutic composition directly to the tumor site (e.g., through a surgically implanted catheter).

Alternatively, the therapeutic composition can be placed at the target site in a slow release formulation. Such formulations can include, for example, a biocompatible sponge or other inert or resorbable matrix material impregnated with the targeted nanoparticles, slow dissolving time release capsules or microcapsules, and the like.

Typically the catheter or time release formulation will be placed at the tumor site as part of a surgical procedure. Thus, for example, where major tumor mass is surgically removed, the perfusing catheter or time release formulation can be emplaced at the tumor site as an adjunct therapy. Of course, surgical removal of the tumor mass may be undesired, not required, or impossible, in which case, the delivery of the therapeutic compositions of this invention may comprise the primary therapeutic modality.

18. Kits

In various embodiments, this invention provides kits for the practice of this invention. The kits can comprise one or more containers containing superparamagnetic nanoparticles as described herein. The nanoparticles can optionally be surface coated or otherwise treated to allow for intracellular introduction. The kit is preferably designed so that the manipulations necessary to perform the desired reaction should be as simple as possible to enable the user to prepare from the kit the desired composition by using the facilities that are at his disposal. Therefore, the invention also relates to a kit for preparing a composition according to this invention. In certain embodiments, the kit can optionally, additionally comprise a reducing agent and/or, if desired, a chelator, and/or instructions for use of the composition and/or a prescription for reacting the ingredients of the kit to form the desired product(s). If desired, the ingredients of the kit may be combined, provided they are compatible.

When kit constituent(s) are used as component(s) for pharmaceutical administration (e.g. as an injection liquid) they are preferably sterile and can, optionally be provided in a pharmacologically acceptable excipient. When the constituent(s) are provided in a dry state, the user should preferably use a sterile physiological saline solution as a solvent. If desired, the constituent(s) can be stabilized in the conventional manner with suitable stabilizers, for example, ascorbic acid, gentisic acid or salts of these acids, or they may comprise other auxiliary agents, for example, fillers, such as glucose, lactose, mannitol, and the like.

In certain embodiments, the kits additionally comprise instructional materials teaching the use of the compositions described herein (e.g., nanoparticles, derivatized nanoparticles, etc.) in electron spin resonance applications for selectively heating cells, tissue, organs, and the like.

While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

19. REFERENCES

[1] Rossi-Fanelli A., Cavaliere R., Mondovi B., Moricca G., “Selective Heat Sensitivity of Cancer cells”, p 1-185, Recent Results in Cancer Research, Springer-Verlag, Berlin Heidelberg, New York, (1977).
[2] Simard R., Bernhard W., “A heat-Sensitive Cellular Function Located in the Nucleolus”, Journal of Cell Biology 34, 61 (1967).
[3] Warocquier R., Scherrer K., “RNA Metabolism in Mammalian Cells at Elevated Temperature”, European Journal of Biochemistry 10, 362 (1969).
[4] S. K. Alpard et al, Perfusion 11, 425 (1996).
[5] H. Shidnia et al, Oncology 50, 353 (1993).
[6] Henderson M A, Pettigrew R T, Induction of Controlled Hyperthermia in Treatment of Cancer, p 1275, Lancet (1971).
[7] Stehlin J S, Giovanella B C, de Ipolyi P D, Muenz L R, Anderson R F, “Results of Hyperthermic Perfusion for Melanoma of the Extremities”, Surgery, Bynecology, and Obstetrics 140, 339 (1975).
[8] R. T. Gordon, J. R. Hines, D. Gordon, Med. Hypotheses 5, 83 (1979).
[9] A. Jordan et al, J. Magn. Magn. Mater. 201, 413 (1999).
[10] The Encyclopaedia of Medical Imaging, volume III.
[11] M. H. Falk and R. D. Issels, Int. J. Hyperthermia 17, 1 (2001).
[12] N. A. Brusentsov et al, J. Magn. Magn. Mater. 225, 113 (2001).
[13] A. Jordan et al, J. Magn Magn. Mater. 225, 118 (2001).
[14] M. Yanase et al, Jpn. J. Cancer Res. 89, 463 (1998).
[15] B. Leetal, J. Chem. Eng. Jpn. 34, 66(2001).
[16] A. Jordan et al, Int. J. Hyperthermia 13, 587 (1997).
[17] M. Shinkai et al, Jpn. J. Hyperthermic Oncol. 14, 15 (1998).
[18] L. Néel, Ann. Geophys. 5, 99 (1949).
[19] Y. Rabin, Int. J. Hyperthermia 18, 194 (2002).
[20] O. Zohar, M. Ikeda, H. Shinagawa, H. Inoue, H. Nakamura, D. Elbaum, D. L. Alkon, and T. Yoshioka, Biophy. J. 74, 82 (1998).
[21] C. P. Poole, Electron Spin Resonance (2nd Edition), Wiley-Interscience, (1983).
[22] E. E. Anderson, J. R. Cunningham Jr., G. E. McDuffie, and R. F. Stauder, J. Phys. Soc. Japan 17, 365 (1962).
[23] I. Brigger, C. Dubernet, P. Couvreur, Adv Drug Deliv Rev. 54, 631-651. (2002).
[24] M. Lewin et al, Nat. Biotechnol. 18, 410-414 (2000).
[25] E. A. Schellenberger et al, Mol. Imaging. 1,102 (2002).
[26] P. Wunderbaldinger et al, Acad Radiol. 9, S304 (2002).
[27] Y. X. Wang, S. M. Hussain, G. P. Krestin, Eur. Radiol. 11, 2319 (2001).
[28] Y. Zhang, N. Kohler and M. Zhang, Biomaterials 23, 1553 (2002).
[29] G. F. Dionne, JAP 41, 4874 (1970).
[30] S. Knorr, A. Grupp, M. Mehring, M. Waiblinger, and A. Weidinger, AIP Conference Proceedings 544, 191 (2000).
[31] X.-D. Xiang, X. Sun, G. Briceno, Y. Lou, K.-A. Wang, H. Chang, W. G. Wallace-Freedman, S.-W. Chen, P. G. Schultz, Science 268, 1738 (1995).
[32] G. Bricefio, H. Chang, X. Sun, P. G. Schultz, X.-D. Xiang, Science 270, 273 (1995).
[33] X. Sun, K. A. Wang, Y. Yoo, W. G. Wallace-Freedman, C. Gao, X. D. Xiang, P. Schultz, Advanced Materials 9, 1046 (1998).
[34] H. Chang, C. Gao, I. Takeuchi, Y. Yoo, J. Wang, P. G. Schultz, X.-D. Xiang, R. P. Sharma, M. Downes, App. Phys. Lett. 72, (1998).
[35] W. R. Cannon et al, Journal of the American Ceramic Society 65, 324 (1982).
[36] M. A. Garstens and J. I. Kaplan, Phys. Rev. 99, 459 (1955).
[37] M. A. Garstens, Phys. Rev. 93, 1228 (1954).
[38] M. A. Garstens, L. S. Singer, and A. H. Ryan, Phys. Rev. 96, 53 (1954).
[39] R. Hergt, W. Andrä, C. G. d'Ambly, I. Hilger, W. A. Kaiser, U. Richter, and H.-G. Schmidt, IEEE Transactions on Magnetics 34, 3745 (1998).
[40] A. Jordan et. al., J. Magn. and Magn. Mater. 194, 185 (1999).
[41] B. Arkles, CHEMTECH 7, 766 (1977).
[42] R. S. Molday, and D. Mackenzie, J. of Immunological Methods 52, 353 (1982).
[43] H. Pardoe et. al., J. Magn. Magn. Mater. 225, 41 (2001).
[44] V. S. Zaitsev et. al., J. Colloid Interface Science 212, 49 (1999).
[45] P. K. Gupta et. al., International Journal of Pharmaceutics 43, 167 (1988).
[46] D. Muller-Schulte and D. Brunner, Journal of Chromatography A 711, 53 (1995).
[47] E. Bourgeat-Lami and J. J. Lang, J. Colloid Interface Science 197, 293 (1998).
[48] R. V. Upadhyay, D. Srinivas, R. V. Mehta, J. Mag. Mag. Mater. 214, 105 (2000).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition for selectively heating a cell or tissue intracellularly, said composition comprising:
a superparamagnetic nanoparticle treated to be incorporated intracellularly into said cell or tissue.

2. The composition of claim 1, further comprising:
a mixture of compositions each selected for one or more of:
(1) selectively heating a cell;
(2) imaging a cell, tissue, or organ; and
(3) providing internal thermometry in a cell, tissue or organ.

3. The composition of claim 1, wherein said superparamagnetic nanoparticle comprises a material selected from the group consisting of:
materials with an electron spin resonance (ESR) Q greater than than 10;
materials with an electron spin resonance (ESR) Q ranging from about 100 to about 1000.
a garnet or a spinel.
a garnet or a spinel selected from Table 2;
yttrium ion garnet (YIG).

4-8. (canceled)

9. The composition of claim 1, wherein said superparamagnetic nanoparticles further comprise surface modifications allowing for bio-compatible solutions, said modifications selected from the group consisting of:
coating with aminosilane or silane;
hydrolysis of the three labile groups of (MeO)₃SiCH₂CH₂CH₂NH₂. 2% w/v of (MeO)₃SiCH₂CH₂CH₂NH₂ is to be dissolved in de-ionized water under ultrasonic mixing conditions for several minutes to formulate (OH)3SiCH2CH2CH2NH2.
condensation of (OH)₃SiCH₂CH₂CH₂NH₂ to formulate oligomers as follows: (OH)₂Si(R)—O—(R)Si(OH)—O—Si(R)(OH)₂, R═CH₂CH₂CH₂NH₂ via ultrasonic mixing for another five to ten minutes;
use a pre-prepared colloidal nano-particle (YIG) solution without aggregates wherein said colloidal solution's pH is adjusted by ammonium hydroxide to keep the pH at 8-9;
mix said YIG nano-particle colloidal solution with the Si oligomers solution under ultrasonic to form hydrogen bonds between the OH groups of nano-particles and of the Si oligomers;
allow a covalent linkage to form with the substrate by loss of water to form Fe—O—Si bonds under ultrasonic mixing and at temperatures around 60° C.
isolate a solution of nano-particles (YIG) with aminosilane shells from uncoated polymers and MeOH through gel filtration chromatography.
preparing said nanoparticles with a Dextran type shell.

10-12. (canceled)

13. The composition of claim 9, wherein said superparamagnetic nanoparticle has at least one dimension less than about 500 nm.

14-19. (canceled)

20. A method of selectively heating a cell, tissue, or molecule, said method comprising:
contacting said cell, tissue, or molecule with a composition comprising a superparamagnetic nanoparticle, a ferromagnetic nanoparticle, or a ferimagnetic nanoparticle that are selectively taken up by a biological target comprising said cell, tissue, or molecule; and
heating said superparamagnetic nanoparticle intracellularly on a microscopic scale using electron spin resonance and/or ferromagnetic resonance to selectively thermally treat cells containing said composition.

21. The method of claim 20, wherein said electron spin resonance is at an RF ranging from about RF frequency ranging from 200 to 2,000 MHz.

22. (canceled)

23. The method of claim 20, wherein said electron spin resonance is spatially localized by a magnetic field gradient over a region smaller than the region over which the superparamagnetic nanoparticles are distributed.

24-30. (canceled)

31. A method of treating cancer cells comprising:
using a composition of nano-particles and ferromagnetic resonance for intracellular cancer thermal ablation therapy;
using said composition and said resonance for internal thermometry; and
selecting nano-particles that are predominately ingested by targeted cancer cells rather than by normal cells.

32-34. (canceled)

35. The method of claim 31 further comprising:
controlling an applied local magnetic field and an electromagnetic radiation frequency to direct ferromagnetic resonance and heating to a specific volume at a specific location to thereby only heat and kill cells ingested with nano-particles at a specific location.

36. The method of claim 31 further comprising:
using the temperature dependence of ferromagnetic electron resonance frequencies of said composition as internal thermometry to monitor the temperature of said particles and said cells.

37. The method of claim 31 further comprising:
using said nano-particles as MRI imaging and/or eMRI contrast agents to enable MRI image guided surgical heating therapy.

38. The method of claim 31 further wherein:
densely packed nano-particles (and optionally proteins) within cancer cells are used to heat those cells to a much higher temperature than the average temperature of the region, especially the temperature of normal cells with no nano-particle filling.

39-42. (canceled)

43. The method of claim 31 further wherein:
a temperature of the target (e.g. tumor cells or region) can be raised by ˜10° C. within several seconds.

44-49. (canceled)

50. The method of claim 31 further comprising:
using ESR based imaging thereby allowing heat treatment, imaging and internal thermometry with same equipment at lower cost than conventional MRI because the required magnetic field for ESR is very low (<500 Gauss).

51. (canceled)

52. The method of claim 31 further comprising:
using different materials in said composition, one or more selected for efficient heating and one or more selected for temperature dependence of electron spin resonance frequency to allow for thermometry applications.

53. The method of claim 31 further wherein:
said composition comprises synthesized Ga doped YIG SPM nano-particles suspended in bio-compatible solution with ferromagnetic resonance below 1 GHz and heat absorption efficiency at least one order of magnitude higher than Néel heating media.

54-59. (canceled)

60. A method of selectively heating a cell, tissue, or organ, said method comprising:
delivering intracellularly a plurality of superparamagnetic nanoparticles into one or more cells that comprise said cell, tissue, or organ; and
heating said superparamagnetic nanoparticles using electron spin resonance.

61. The method of claim 60, wherein said superparamagnetic nanoparticles are delivered directly into said cell, tissue, or organ by injection or via a catheter or during a surgical procedure.

62-66. (canceled)

67. The method of claim 60, wherein said electron spin resonance is spatially localized by a magnetic field gradient over a region smaller than the region over which the superparamagnetic nanoparticles are distributed.

68-73. (canceled)

74. The method of claim 60, further comprising imaging said cell, tissue, or molecule using a method selected from the group consisting of thermography, MRI, ESR, and x-ray.

75. The method of claim 60, wherein said cell or tissue is a cancer cell.

76-78. (canceled)

79. A kit for selectively heating or imaging a cell or tissue, said kit comprising:
a container containing a composition of superparamagnetic nanoparticles of claim 1 prepared for intracellular uptake to a biological target comprising said cell or tissue.

80-85. (canceled)
